(19) United States
(12) Patent Application Publication
(10) Pub. No.: US 2010/0221367 A1
(43) Pub. Date: Sep. 2, 2010

(54) USE OF SOLIDAGO VIRGAUREA IN THE TREATMENT AND PREVENTION OF VIRAL INFECTIONS

(76) Inventor: Shirong LOU, Hangzhou (CN)

Correspondence Address: MATTHIAS SCHOLL, 14781 MEMORIAL DRIVE, SUITE 1319, HOUSTON, TX, 77079, US

(21) Appl. No.: 12/781,041

(22) Filed: May 17, 2010

Related U.S. Application Data
(63) Continuation-in-part of application No. 11/970,589, filed on Jan. 8, 2008.

(30) Foreign Application Priority Data
Sep. 7, 2006 (CN) 200610127149.1
Sep. 7, 2006 (CN) 200610127150.4
Jul. 6, 2007 (CN) PCT/CN2007/002084

(51) Int. Cl. A61K 36/315; A61K 36/28; A61K 36/484; A61P 31/16

(52) U.S. Cl. 424/755; 424/764; 424/757

(57) ABSTRACT

A method for treating H5N1 avian influenza caused by H5N1 avian influenza virus in an animal by administering to an animal suffering from H5N1 avian influenza, a pharmaceutical composition of Solidago virgaurea. The pharmaceutical composition of Solidago virgaurea comprises an herbal stock solution extracted from the natural plant Solidago virgaurea. The herbal extract has anti-H5N1 avian influenza and anti-influenza properties. The pharmaceutical composition of Solidago virgaurea may further contain excipients, adjuvants or carriers.

### CROSS-REFERENCE TO RELATED APPLICATIONS

This application is a continuation-in-part of U.S. Ser. No. 11/970,589 filed on Jan. 08, 2008, now pending, which is a National Stage Application of International Patent Application No. PCT/CN2007/002084 with an international filing date of Jun. 7, 2007, designating the United States, now pending, and further claims priority benefits to Chinese Patent Application No. 200610127149.1 filed on Sep. 7, 2006, and to Chinese Patent Application No. 200610127150.4 filed on Sep. 7, 2006. The contents of the aforementioned specifications are incorporated herein by reference.

### BACKGROUND OF THE INVENTION

1. Field of the Invention

This invention relates to the use of Solidago virgaurea in the treatment and prevention of the H5N1 avian influenza and to lowering of the concentration of the H5N1 avian influenza virus in an animal, e.g., a human.

2. Description of the Related Art

Influenza A virus subtype H5N1, also known as A (H5N1) or simply H5N1, is a subtype of the Influenza A virus which can cause illness in humans and many other animal species. A bird-adapted strain of H5N1, called HPAI A (H5N1) for “highly pathogenic avian influenza virus of type A of subtype H5N1”, is the causative agent of H5N1 flu, commonly known as “avian influenza” or “bird flu”. Influenza is a commonly occurring disease that has not been overcome till now, and the difficulty of treating influenza lies in the ongoing mutations of the virus. Because humans are facing the threat of mutation of H5N1 highly pathogenic avian influenza, there is an urgent need to develop effective drugs that can resist influenza virus ongoing mutations and that of various other types of influenza viruses.

According to pharmacopoeia records, the use of Solidago virgaurea in the Chinese traditional medicine is mainly to dispel wind, remove heat, subdue swelling, detoxicate, treat coldness and headache, sore throat, jaundice, pertussis, children infantile convulsion, traumatic injury, sores of the back, and tinea manuum. Moreover, Solidago virgaurea has apparent bacteriostatic and bacteriocidal properties with respect to staphylococcus aureus, pneumococcus, pseudomonas aeruginosa, shigella flexneri, etc.

### SUMMARY OF THE INVENTION

Thus, according to one embodiment of the invention, there provided is a method for treating and/or preventing H5N1 avian influenza or influenza virus with a pharmaceutical composition comprising Solidago virgaurea.

In certain classes of the embodiments, the pharmaceutical composition comprises 50-100 wt. % Solidago virgaurea.

In certain classes of the embodiments, the pharmaceutical composition is prepared as follows: placing a single material of Solidago virgaurea and a solvent with a weight ratio of 1:50-100 into an extractor, boiling for 10-30 minutes, filtering, purifying the solution according to conventional methods to yield an herbal stock solution, and preparing into an oral liquid or a granule.

In certain classes of the embodiments, the pharmaceutical composition is prepared as follows: placing a single material of Solidago virgaurea and a solvent with a weight ratio of 1:10-100 into an extractor, boiling for 10-30 minutes, filtering, purifying the solution according to conventional methods to yield an herbal stock solution, and preparing into an oral liquid or a granule.

In certain classes of the embodiments, the solvent is ethanol.

In certain classes of the embodiments, the solvent is water.

In certain classes of the embodiments, the pharmaceutical composition comprises 50-90 wt. % of Solidago virgaurea and 10-50 wt. % of glycyrrhiza.

In certain classes of the embodiments, the pharmaceutical composition comprises 50-90 wt. % of Solidago virgaurea, 5-25 wt. % of honeysuckle, and 5-25 wt. % of radix isatidis.

In accordance with another embodiment of the invention, provided is a pharmaceutical composition comprising Solidago virgaurea, wherein the pharmaceutical composition is provided in the form of a granule, a capsule, or a herb decoction piece through micron-milling a material comprising Solidago virgaurea, or in the form of a tea package prepared through milling by common disintegrator, and comprises 50-100 wt. % of Solidago virgaurea.

In certain classes of the embodiments, the pharmaceutical composition comprises 50-90 wt. % of Solidago virgaurea and 10-50 wt. % of glycyrrhiza.

In certain classes of the embodiments, the pharmaceutical composition comprises 50-90 wt. % of Solidago virgaurea, 5-25 wt. % of honeysuckle, and 5-25 wt. % of radix isatidis.

In accordance with still another embodiment of the invention, provided is a method for treating and/or preventing H5N1 avian influenza or influenza with a pharmaceutical composition comprising Solidago virgaurea, wherein the pharmaceutical composition is provided in the form of an injection preparation, a granule, a spray, a capsule, or a dropping pill after purifying a material comprising Solidago virgaurea through supercritical CO₂ liquid extraction, and comprises 50-100 wt. % of Solidago virgaurea.

In certain classes of the embodiments, the pharmaceutical composition comprises 50-90 wt. % of Solidago virgaurea and 10-50 wt. % of glycyrrhiza.

In certain classes of the embodiments, the pharmaceutical composition comprises 50-90 wt. % of Solidago virgaurea, 5-25 wt. % of honeysuckle, and 5-25 wt. % of radix isatidis.

In certain classes of the embodiments, Solidago virgaurea is an entire Solidago virgaurea plant.

### DETAILED DESCRIPTION OF THE EMBODIMENTS

Conventional purification methods of Solidago virgaurea are as follows.

(1) The common apparatus for the purification of technology of the water extract: sedimentation tank, centrifuge, ultrafilter and ultrafiltration membrane etc.

(2) Technology process: According to the theory that solid particle can be separated from the liquid in the liquid medium by natural sedimentation due to the gravity, in the sedimentation separation technology, put the water extract into the sedimentation tank standing for a period of time, and then extract the upper pellucid liquid with siphon, also temperature reduction and the addition of sedimentation agent—electrolyte (such as alum) can be used to achieve high efficiency and high quality if necessary.

The existing micron-milling wall-braking technique:

It is completed by the use of the device combining the nanometer milling and wall-broken technologies called “air flow pulverizer” developed by Angli Natural Drug Engineering Technology Ltd. of Shanghai Jiaotong University.

The theory is that: after the materials enter the milling room of air flow pulverizer in which there are several milling nozzles, the supersonic flow of the milling nozzles gets the milled materials impacted by the high-speed air flow and the mutual collision among the powders, and due to the high speed air flow and strong impact force, the milling effect can reach 0.5-10 micron-size particles (500 nm-10000 nm) and realize physical plant cell wall breaking to make the human body absorb the effective materials completely.

The existing supercritical CO₂ liquid extraction technique:

It is completed by “1000 L automatic large scale supercritical units” developed by Shenyang DongYu Corp. with high extraction capability, high extraction rate and controllable quality. The theory is that: the critical temperature and pressure of CO₂ are respectively 31.05° C. and 7.38 MPa, and CO₂ has double characteristics of gas and liquid when above the critical point. It is approximate to gas with similar viscosity to gas; as well it is approximate to liquid with similar density to liquid, but much larger diffusion coefficient than liquid. Meanwhile, it is a good solvent, and can dissolve many materials by the mutual function and diffusion function among the molecular. At the same time, in the regions higher than the critical point, pressure has a little change, which can cause great changes of its density so as to bring larger changes of the solubility. Therefore, supercritical CO₂ can dissolve the materials in the matrix, forming the supercritical CO₂ load phase, then reducing the pressure of the carrier gas or rising temperature, the solubility of supercritical CO₂ is reduced, and these materials are deposited to be separated from CO₂ (resolution) so as to realize the extraction and separation goal.

The used method and effective dose of the drugs in this invention for treating H5N1 avian influenza or influenza virus are listed as follows:

The oral dose of the herbal stock solution is 2-4 times per day and 100-300 mL per time for the adults.

For the oral liquid, the oral dose is 2-4 times per day and 10-20 mL per time for adults.

For the granule, capsules or decoction pieces, the dose is 3-4 times per day and 2-6 g per time for adults.

For the injection, the dose is twice per day and 5-10 mL per time for adults.

For the dropping pill, the dose is three times per day and 8 pills per time for adults.

For the spray sprayed in the mouth, the dose is several times per day.

According to the records in page 8 to page 9 of the Dictionary of Traditional Chinese drugs (first volume) (edited by the Jiangsu New College of Medicine), the Solidago virgaurea is a composite plant; its whole herb or the whole herb with root are usually used; it is bitter and cool taste. The whole herb Solidago virgaurea contains hydroxybenzene ingredient, tannin, volatile oil, saponin, and flavonoids, etc. Its main functions are mainly in dispelling wind and cleaning heat, subduing swelling and detoxication, treating cold and headache, sore throat, jaundice, pertussis, children infantile convulsion, traumatic injury, carbuncle on the back and tinea manuum. Moreover, it has obvious killing and inhibition functions on staphylococcus aureus, pneumococcus, pseudomonas aeruginosa and shigella flexneri etc.

The medical efficiency of Solidago virgaurea is in dispelling wind and cleaning heat, diminishing inflammation and detoxicating; its pharmacological functions can reach liver and gallbladder meridians with cool medical property.

Glycyrrhiza can purge fire, detoxify and moisten lung to stop coughing; its pharmacological functions can reach all the twelve meridians; it has mild medical nature and can be in concordance with other drugs.

The medical efficiency of honeysuckle is in cleaning heat and detoxicating, diminishing inflammation and alleviating pain; its pharmacology functions into lung, stomach and colorectal meridians; and cold medical property.

The medical efficiency of radix isatidis is in cooling blood and clearing fire, diminishing inflammation and analgesia; its pharmacology functions into heart and lung meridians; and cold medical property.

The combination of Solidago virgaurea and glycyrrhiza can regulate the medical property of Solidago virgaurea and enhance the adaptability.

The compatibility of Solidago virgaurea with honeysuckle and radix isatidis can improve the attending efficiency and increase the broad-spectrum property.

This inventor with decades of clinical experiences validates that the herbal stock solution not only has obvious killing and inhibition functions on H5N1 avian influenza or influenza virus, but also has very good variation resistance.

This invented drug is used for treating and preventing H5N1 avian influenza or influenza virus, wherein the efficiency rate is above 90%.

The following statistical reports of clinical observation in curative effect are used to further validate the effects of this invention.

1. Clinical Data:

Treating and preventing various types of influenza:

1) Using Solidago virgaurea extract with concentration of 1:100 to treat 513 cases with influenza A, 2-4 times of oral administrations for patients, 300 mL per time for adults and about 20-300 mL for children according to their ages. Wherein, 399 cases were cured within one day, accounting for 77.8%; 92 cases were cured within two days, accounting for 17.9%; 22 cases were cured within three days, accounting for 4.3%. The total effective rate was 100%.

2) Using Solidago virgaurea extract with concentration of 1:100 to treat 253 cases with influenza B, 2-4 times of oral administrations for patients, 300 mL per time for adults and about 20-300 mL for children according to the age. Wherein, 165 cases were cured within one day, accounting for 65.2%; 46 cases were cured within two days, accounting for 18.2%; 25 cases were cured within three days, accounting for 9.9%; 17 cases were inefficient, accounting for 6.7%. The total effective rate was 93.3%.

3) Using Solidago virgaurea extract with concentration of 1:100 to treat 116 cases with influenza C, 2-4 times of oral administrations for patients, 300 mL per time for adults and about 20-300 mL for children according to the age. 51 cases were cured within one day, accounting for 44%; 27 cases were cured within two days, accounting for 23.3%; 28 cases were cured within three days, accounting for 24.1%; 10 cases were inefficient, accounting for 8.6%. The total effective rate was 91.4%.

4) During the period of influenza, 1291 healthy persons took Solidago virgaurea extraction with the concentration of 1:100 to prevent the influenza virus infection, one time per day, 60-150 mL for adults and 20-80 mL for children. The results proved that the effective prevention rate was 100%.

Treating undefined pneumonia: On December 2003 and September 2004, 2 cases with undefined pneumonia were cured using Solidago virgaurea extract with concentration of 1:100. The patients were both adults; they were the patients in ICU in Zhejiang Tongde Hospital and the First Affiliated Hospital of Zhejiang Medical University, respectively; they both had sustaining hyperpyrexia (above 39° C.) for more than seven days with rapid disease development, appearing pulmonary hemorrhage, pleural effusions, dyspnea and pulmonary signs of consolidation; SARS virus infection was excluded with laboratory RNA and PCR detections, however, virus could be extracted from tracheal exsuction materials of the patients; the chest image detected serious lung infusion, showing consolidation image of lungs with large ground-glass shape. Both hospitals sent “Notice of Critical Illness”. Under this condition, patient relatives asked for help via relations. Solidago virgaurea extract was used with the concentration of 1:100 to treat these two patients, 3-4 times per day, 300-500 mL per time and sequential administration for three days. The body temperature of the patients reduced to below 37.8° C. within two days with rapid spirit recovery; the life signs obviously became better, and then entered the pneumonia recovery period in the common sickroom. They left hospital after about 15 days with good prognosis.

Treating and preventing H5N1 avian influenza: On 13 Jan. 2004, a large number of chicken died in the farm in Dongting village, Guangde county, Anhui province. Using the goldenrod extract with the concentration of 1:60 to cure 584 infected chickens from 16 January, 40-60 mL of oral dose for each chicken, adding another administration after two hours; feeding 242 healthy chicken respectively with the fine feedstuff mixed in a ratio of 1:4 with the goldenrod extract with the concentration of 1:100, and with the fine feedstuff mixed in a ratio of 1:10 with the goldenrod powder. According to the observation results after two hours, four hours, ten hours, 30 hours, 60 hours and five days, except 26 chicken died during initial administration period (within five minutes), other chicken all recovered with normal activities; the effective rate was above 95%; all healthy chicken were protected efficiently, and protection effective rate was up to 100%. The participating workers and chicken farm workers drank a cup of 300 mL of Solidago virgaurea extract with the concentration of 1:100 to prevent virus infection, and they all retain health at the end of experiment (At the beginning of Feb, the National Disease Controlling Center determined that the epidemic broken out in Guangde country of Anhui was H5N1 highly pathogenic avian influenza).

2. The diagnosis basis is GB 15994-1995 Diagnosis Criteria for Influenza and GB/T18936-2003 Diagnosis Technique for Highly Pathogenic Avian Influenza.

3. The treatment methods: treating influenza: 10 g of Solidago virgaurea is boiled in 1000 mL of water for ten minutes, the obtained 900 mL of herbal stock solution in this invention is taken by patients; 2-4 times per day, and 300 mL per time. Treating avian influenza with 10 g of Solidago virgaurea plus 600 mL of water, oral administration for 2-4 times per day, 40-60 mL per time.

4. The evaluation standards for curative effect: 1) patients feel better: body temperature reduces to below 37.5° C., dry cough is alleviated, with smooth breath; Cure: body temperature changes to normal, dry cough disappears; pneumonia recovers well, with normal breath. 2) The spirits of ill avian get well, with normal activities.

5. Curative effect observation: After the administration for three days, the spirit and mood of patients got better, with normal body temperature and well prognosis; finally the patients were cured. The ill avian had good spirits and appetites after five-day observation.

6. Conclusion: using the herbal in this invention to treat and prevent H5N1 influenza or influenza virus, the effective rate is above 90%.

The following examples are used to further explain but not to limit the invention.

### EXAMPLE 1

100 g of Solidago virgaurea and 5,000 mL of water were transferred into the extractor, after boiling for 10 minutes, 4,800 mL of the herbal stock solution was obtained. Oral liquids or decoction pieces could be prepared according to the conventional method after filtering and concentrating.

### EXAMPLE 2

100 g of Solidago virgaurea and 10,000 mL of water were transferred into the extractor, after boiling for 25 minutes, 9,500 mL of the herbal stock solution was obtained. Oral liquids or decoction pieces could be prepared according to the conventional method after filtering and concentrating.

### EXAMPLE 3

90 g of Solidago virgaurea, 10 g of glycyrrhiza and 5,000 mL of water were transferred into the extractor, after boiling for 30 minutes and filtering, 4400 mL of the herbal stock solution was obtained.

### EXAMPLE 4

50 g of Solidago virgaurea, 25 g of honeysuckle, 25 g of radix isatidis and 5,000 mL of water were transferred into the extractor, after boiling for 30 minutes and filtering, 4,400 mL of the herbal stock solution was obtained.

### EXAMPLE 5

100 g of Solidago virgaurea was used to prepare granules, capsules or decoction pieces in this invention after ultrafine milling according to the conventional method.

### EXAMPLE 6

90 g of Solidago virgaurea and 10 g of glycyrrhiza were used to prepare granules, capsules or decoction pieces in this invention after ultrafine milling according to the conventional method.

### EXAMPLE 7

50 g of Solidago virgaurea, 25 g of honeysuckle and 25 g of radix isatidis were used to prepare granules, capsules or decoction pieces in this invention after ultrafine milling according to the conventional method.

### EXAMPLE 8

100 g of Solidago virgaurea was used to prepare injections, granules, sprays, capsules or dropping pills in this invention after supercritical CO₂ fluid extraction according to the conventional method.

### EXAMPLE 9

90 g of Solidago virgaurea and 10 g of glycyrrhiza were used to prepare injection, granules, sprays, capsules or dropping pills in this invention after supercritical CO₂ fluid extraction according to the conventional method.

### EXAMPLE 10

50 g of Solidago virgaurea, 25 g of honeysuckle and 25 g of radix isatidis were used to prepare injection, granules, sprays, capsules or dropping pills in this invention after supercritical CO₂ fluid extraction according to the conventional method.

The invention claimed is:

1. A method for treating H5N1 avian influenza caused by H5N1 avian influenza virus in an animal comprising administering to an animal suffering from H5N1 avian influenza, a pharmaceutical composition comprising Solidago virgaurea.

2. The method of claim 1, wherein said pharmaceutical composition comprises 50-100 wt. % Solidago virgaurea.

3. The method of claim 1, wherein said pharmaceutical composition is prepared as follows: placing a single material of Solidago virgaurea and a solvent with a weight ratio of 1:10-100 into an extractor, boiling for 10-30 minutes, filtering, purifying the solution according to conventional methods to yield an herbal stock solution.

4. The method of claim 3, wherein said solvent is water.

5. The method of claim 3, wherein said solvent is ethanol.

6. The method of claim 1, wherein said pharmaceutical composition is provided in the form of an oral liquid, a granule, a decoction piece, a capsule, spray, a pill, an injection, or freeze dried powder.

7. The method of claim 1, wherein Solidago virgaurea is an entire Solidago virgaurea plant.

8. The method of claim 1, wherein said pharmaceutical composition comprises 50-90 wt. % of Solidago virgaurea and 10-50 wt. % of glycyrrhiza.

9. The method of claim 1, wherein said pharmaceutical composition comprises 50-90 wt. % of Solidago virgaurea, 5-25 wt. % of honeysuckle, and 5-25 wt. % of radix isatidis.

10. The method of claim 1, wherein said pharmaceutical composition is provided in the form of a granule, a capsule, or a herb decoction piece prepared through micron-milling technology, or in the form of a tea package prepared through milling by common disintegrator, and comprises 50-100 wt. % of Solidago virgaurea.

11. The method of claim 1, wherein said pharmaceutical composition is provided in the form of a granule, a capsule, or a herb decoction piece prepared through micron-milling technology, or in the form of a tea package prepared through milling by common disintegrator, and comprises 50-90 wt. % of Solidago virgaurea and 10-50 wt. % of glycyrrhiza.

12. The method of claim 1, wherein said pharmaceutical composition is provided in the form of a granule, a capsule, or a herb decoction piece prepared through micron-milling technology, or in the form of a tea package prepared through milling by common disintegrator, and comprises 50-90 wt. % of Solidago virgaurea, 5-25 wt. % of honeysuckle, and 5-25 wt. % of radix isatidis.

13. The method of claim 1, wherein said pharmaceutical composition is provided in the form of an injection preparation, a freeze dried powder, a decoction piece, an oral liquid, a granule, a spray, a capsule, or a pill through purifying with supercritical CO₂ fluid extraction, and comprises 50-100 wt. % of Solidago virgaurea.
